(19) United States
(12) Patent Application Publication
(10) Pub. No.: US 2024/0181231 A1
(43) Pub. Date: Jun. 6, 2024

(54) SUBMUCOSAL IMPLANTABLE DRUG DELIVERY APPARATUSES, SYSTEMS, AND METHODS

(71) Applicant: Massachusetts Eye and Ear Infirmary, Boston, MA (US)

(72) Inventor: Benjamin S. Bleier, Weston, MA (US)

(21) Appl. No.: 18/284,637

(22) PCT Filed: Mar. 28, 2022
(86) PCT No.: PCT/US2022/022205

§ 371 Date: Sep. 28, 2023

Related U.S. Application Data
(60) Provisional application No. 63/167,415, filed on Mar. 29, 2021.

(51) Int. Cl. A61M 31/00

(52) U.S. Cl. CPC A61M 31/002; A61M 2205/04; A61M 2205/10; A61M 2210/0618

(57) ABSTRACT

The present disclosure relates to systems, apparatuses, and methods of continuous delivery of therapeutic agents. The apparatuses provide an implantable device that can be implanted in a nasal region of a subject to enable continuous administration of a therapeutic agent to the central nervous system (CNS) of the subject. The implantable device for drug delivery can include a distal body having a wall extending from a proximal end to a distal end, and a lumen extending therethrough in a longitudinal direction. The distal body can include one or more apertures in fluid communication with the lumen and located along the wall, and a pair of support members protruding from the body in a transverse direction and extending along the body in the longitudinal direction. The support members can be separated by a gap distance that extends along in the longitudinal direction.

### CLAIM OF PRIORITY

This application claims the benefit of U.S. Provisional Patent Application No. 63/167,415, filed on Mar. 29, 2021, which is incorporated by reference herein in its entirety.

### TECHNICAL FIELD

The subject matter disclosed herein related generally to the field of drug delivery in a nasal region (e.g., submucosal) of an anatomy.

### BACKGROUND

Neurological disorders affecting the central nervous system (CNS) are increasingly recognized as major causes of death and disability worldwide. As the population continues to age, the incidence and health care costs associated with neurological disorders are projected to rise considerably. The difficulty in treating neurological disorders is at least partially attributable to the presence of the blood-brain-barrier (BBB), which prevents many therapeutic agents from reaching the CNS. It is therefore of interest to develop new devices, systems, and methods for efficient delivery of therapeutic agents to the CNS of patients with neurological disorders.

### SUMMARY

The inventor has discovered that the efficacy of non-invasive delivery of therapeutic agents to the central nervous system is greatly improved through a drug delivery (e.g., intermittent or continuous drug delivery) provided by a submucosal implant in the submucosal space of the olfactory mucosa. The submucosal implant can be surgically implanted using a minimally invasive procedure described herein. Once implanted, the submucosal implant can remain in a patient for an extended period of time (e.g., several weeks, several months, or more). As such, the submucosal implant allows intermittent or continuous dosing over an extended period (e.g., several weeks, several months, or more) of time without having to surgically reenter the submucosal space of the patient. The intermittent or continuous infusion of a drug from the submucosal implant over a prescribed period of time can be facilitated by submucosal catheter delivery via a reservoir fluidly coupled to the implant. By intermittent or continuous dosing in the submucosal space, the barriers to diffusion to the olfactory nerves can be greatly minimized relative to transepithelial delivery and/or depot delivery and the drugs are thereby protected from multiple degradative enzymes present in nasal mucus and from clearance in the nasal cavity via mucociliary clearance.

Accordingly, various embodiments disclosed herein provide apparatuses, systems, and methods for an implant that can deliver a drug directly to the submucosal space of the olfactory mucosa over a prolonged period of time. The apparatuses include an implant that resides in the submucosal space of the olfactory mucosa such that the drug can be delivered in a continuous or intermittent manner to standardize the volume of drug delivery. The apparatuses permit delivery of an array of drug carriers, such as liquid carriers.

In various embodiments, the present disclosure relates to systems and implantable devices that can be placed in a nasal region of a subject to enable continuous administration of a therapeutic agent to the CNS of a subject.

Accordingly, aspects of the present disclosure provide an implantable device for drug delivery, the implantable device comprising a distal body having a wall extending from a proximal end to a distal end, and a lumen extending therethrough in a longitudinal direction, the distal body comprising one or more apertures located along the wall, the apertures being in fluid communication with the lumen; and a pair of support members protruding from the body in a transverse direction and extending along the body in the longitudinal direction, wherein the support members are separated by a gap distance that extends along in the longitudinal direction.

In some embodiments, the support members are substantially parallel to one another in the transverse direction. In some embodiments, the support members extend in the same direction along a transverse plane. In some embodiments, the gap distance is configured to engage tissue during implantation.

In some embodiments, the distal body has a circular cross-section along a transverse plane, and wherein the support members are disposed on a minor arc portion of the circular cross-section. In some embodiments, the minor arc portion of the circular cross-section is less than 25% of a circumference of the circular cross-section.

In some embodiments, the support members have a rectangular shape along the transverse plane. In some embodiments, the support members have a rectangular shape along the longitudinal plane. In some embodiments, the support members is an elongate fin.

In some embodiments, each support member has a height to length ratio of about 0.1 to 1.0. In some embodiments, each of the support members has a length of about 1 mm to about 10 mm. In some embodiments, the gap distance between the pair of support members is about 1 mm to about 3 mm.

In some embodiments, the apertures are located along the wall at about 90 degrees from the support members. In some embodiments, the apertures are located along the wall from about 90 degrees to about 180 degrees from the support members. In some embodiments, the apertures are located along the wall from about 10 degrees to no more than 90 degrees from the support members.

In some embodiments, the body has a length of about 30 mm to about 60 mm. In some embodiments, the body has an outer diameter of about 1 mm to about 3 mm.

In some embodiments, the implantable device comprises a collar coupled at the proximal end of the distal portion, wherein the collar has a wall thickness that is greater than a wall thickness of the distal portion.

Aspects of the present invention provide a system for drug delivery, the system comprising any one of the implantable devices described herein; a drug reservoir; and a tube fluidly connecting the implant to the drug reservoir.

In some embodiments, the drug reservoir comprises a drug refill port fluidly connected to the drug reservoir, the drug refill port configured to receive a drug from an external source and to provide a fluidic path to the drug reservoir. In some embodiments, the drug reservoir is an implantable drug reservoir.

In some embodiments, the system comprises a pump communicably coupled to the drug reservoir and configured to move a drug from the drug reservoir to the implant via the tube. In some embodiments, the pump comprises an additional drug refill port through which the drug reservoir can be filled. In some embodiments, the pump is an implantable pump.

Aspects of the present disclosure provide a method for implanting an implantable device in a nasal region, the method comprising making an incision in skin of a subject; delivering any one of the implantable devices described herein from the incision to a target location in the nasal region; and placing at least a portion of the implantable device within the target location.

In some embodiments, the method comprising forming a tunnel extending from the incision to a target location in the nasal region of the subject, wherein the forming the tunnel includes drilling a hole that extends perpendicularly through a nasion of the subject.

In some embodiments, the method comprising fluidly connecting the implant to a drug reservoir via a tube and implanting the drug reservoir and the tube in the subject, wherein the drug reservoir is implanted in the head, the neck, or the abdominal wall of the subject.

In some embodiments, one or more drugs are continuous delivered from the drug reservoir to the implantable device following implantation of the implantable device.

In some embodiments, the target location includes a submucosal space of an olfactory mucosa of the subject.

The implantable systems, devices, and related methods described herein are directed to continuous delivery of a therapeutic agent to the CNS of the subject that provide several improvements over conventional approaches. Such improvements include, but are not limited to:
(a) Improved patient safety resulting at least in part from extracranial implantable drug delivery systems, devices, and methods that have a low risk of infection and complication compared conventional approaches involving highly invasive and complicated administration routes such as intrathecal or intracerebroventricular administration.
(b) Improved patient compliance resulting at least in part from implantable drug delivery systems, devices, and methods that are minimally invasive and simple to implement.
(c) Continuous and controlled delivery of a therapeutic agent achieved by surgically implanting the drug delivery system using a minimally invasive procedure having minimal cosmetic disruption to the patient.
(d) Improved safety and efficacy resulting at least in part from continuous and controlled delivery of a therapeutic agent that can achieve reduced side effects and improved therapeutic effects compared to intermittent dosing regimens.

Accordingly, provided herein are improved systems, devices, and methods for delivery of a therapeutic agent to the CNS of a subject. Treating a disease of the CNS using any of the systems, devices, and methods described herein is also within the scope of the present disclosure.

Transnasal drug delivery strategies exploit the diffusion of therapeutic agents through the olfactory mucosa, thereby bypassing the BBB and providing a route for BBB-impermeant therapeutics to enter the CNS. For example, the minimally invasive nasal depot (MIND) surgical approach involves implanting a biodegradable depot loaded with a therapeutic agent. The MIND technology is described in US 2021/0378947 and US 2021/0023295, each of which is incorporated herein by reference in its entirety. The systems, devices, and methods described herein involve a non-biodegradable implantable device for continuous and long-term drug delivery.

Any therapeutic agent can be delivered using the systems, devices, and methods described herein. Non-limiting examples of therapeutic agents include small molecules, peptides, proteins (e.g., antibodies), and nucleic acids (e.g., mRNA, siRNA, antisense oligonucleotides).

The following paragraphs include various definitions used in this document.

As used herein, a “subject” refers to a mammal including a human and a domestic or a farm animal (e.g., a dog, a horse, a mouse, a rabbit, a pig, a sheep, a goat, or a cow). In some examples, the subject is a human (e.g., an adult or a child).

As used herein, “tissue” refers to a group of cells having the same function and form in cellular organisms. Non-limiting examples of tissue include connective tissue (e.g., adipose tissue, cartilage, bone, blood), epithelial tissue (e.g., scalp, skin, nasal epithelial tissue), muscle tissue (e.g., smooth muscle, skeletal muscle), and nervous tissue (e.g., brain, spinal cord, nerves).

As used herein, “continuous” refers to uninterrupted delivery of a therapeutic agent for an extended period of time (e.g., more than 1 hour, more than 1 day, more than 1 week). As used herein, “intermittent” refers non-continuous delivery of a therapeutic agent, e.g., delivery at prescribed intervals (e.g., regular intervals or irregular intervals).

As used herein, the term “treating” refers to the application or administration of a composition including one or more active agents to a subject who has a neurological disorder, a symptom of a neurological disorder, and/or a predisposition toward a neurological disorder, with the purpose to cure, heal, alleviate, relieve, alter, remedy, ameliorate, improve, or affect the disorder, the symptom of the neurological disorder, and/or the predisposition toward the neurological disorder.

“Development” or “progression” of a disease means initial manifestations and/or ensuing progression of the disease. Development of the disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence of a neurological disorder includes initial onset and/or recurrence.

“An effective amount” as used herein refers to the amount of each active agent required to confer therapeutic effect on the subject, either alone or in combination with one or more other active agents.

As used herein, “delaying” the development of a neurological disorder means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

The term “about,” as used herein, refers to a number that is +10% of a value that this term precedes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1A is a schematic illustration of a system for drug delivery according to some embodiments described herein.

FIG. 1B is a magnified illustration showing an exemplary implantable device of the system of FIG. 1A in the submucosal space of the olfactory mucosa.

FIG. 1C is an illustration of the implantable device of FIG. 1B within a skull anatomy.

FIG. 1D is a CT image of a subject's head that is superimposed with a schematic illustration of the implantable device of FIG. 1B.

FIG. 2A is a schematic illustration showing a side view of an exemplary implantable device according to some embodiments described herein.

FIG. 2B is a schematic illustration showing a front view of the implantable device of FIG. 2A.

FIG. 3 is a flow chart showing a method for implanting an implantable device in a nasal region according to some embodiments described herein.

FIG. 4 is a schematic illustration showing a subcutaneous drilling device in a nasal region according to certain embodiments described herein.

FIG. 5 is a chart showing uptake of a fluorescently labeled peptide (0.3 mg/kg) in various brain subregions following delivery via the minimally invasive nasal depot (MIND) technique.

FIG. 6 is a chart showing uptake of a fluorescently labeled peptide (0.6 mg/kg) in various brain subregions following delivery via the MIND technique.

FIG. 7 is a chart showing comparison of uptake of different doses of a fluorescently labeled peptide (0.3 mg/kg versus 0.6 mg/kg) in various brain subregions following delivery via the MIND technique.

FIG. 8 is a chart showing comparison of BDNF AntagoNAT (BDNF AT) (0.15 mg/kg) distribution in the rat brain following delivery by ICV, the MIND technique, or IV.

### DETAILED DESCRIPTION

Following below are more detailed descriptions of various concepts related to, and exemplary embodiments of, inventive systems, methods, and components related to continuous drug delivery within a body. In various embodiments, the systems, methods, and components described herein relate to continuous drug delivery to the CNS of a subject.

### Systems for Drug Delivery

Aspects of the present disclosure provide systems for continuous drug delivery to the CNS of a subject. In some embodiments, the systems for drug delivery described herein include an implantable device that is implanted into the nasal region of the subject. The systems described herein can include a drug reservoir that allows storage of a therapeutic agent, and a tube connecting the implantable device to the drug reservoir that provides a pathway for transport of the therapeutic agent from the drug reservoir to the implantable device. In some examples, the system includes a pump (e.g., passive or active pump) fluidly coupled to the drug reservoir and configured to move a drug from the drug reservoir to the implantable device via the tube.

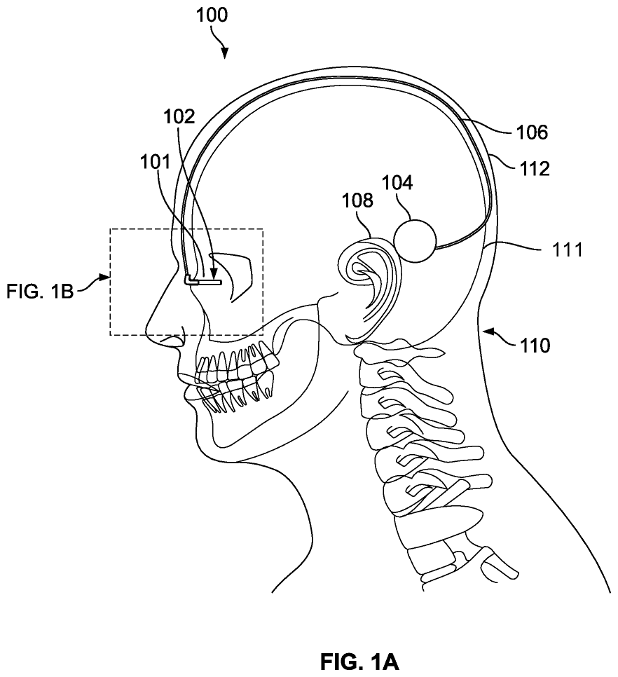

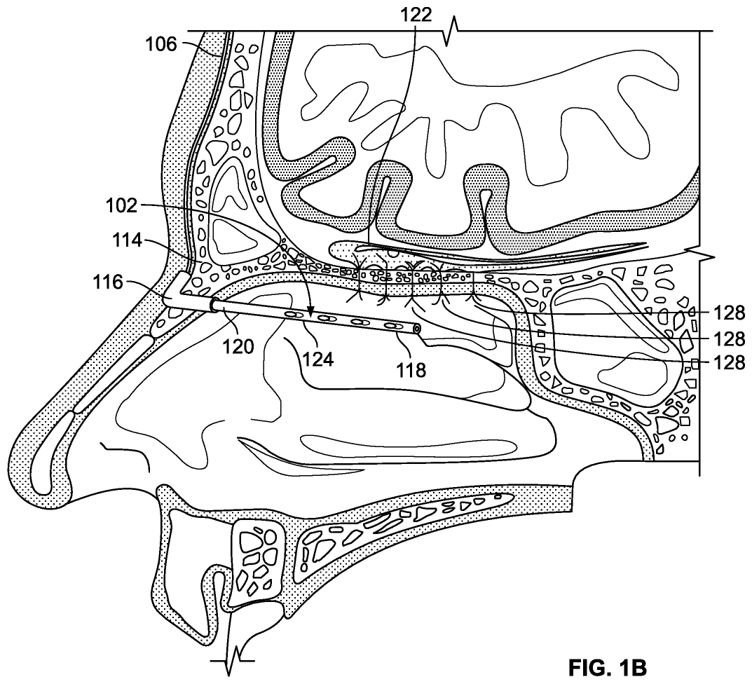

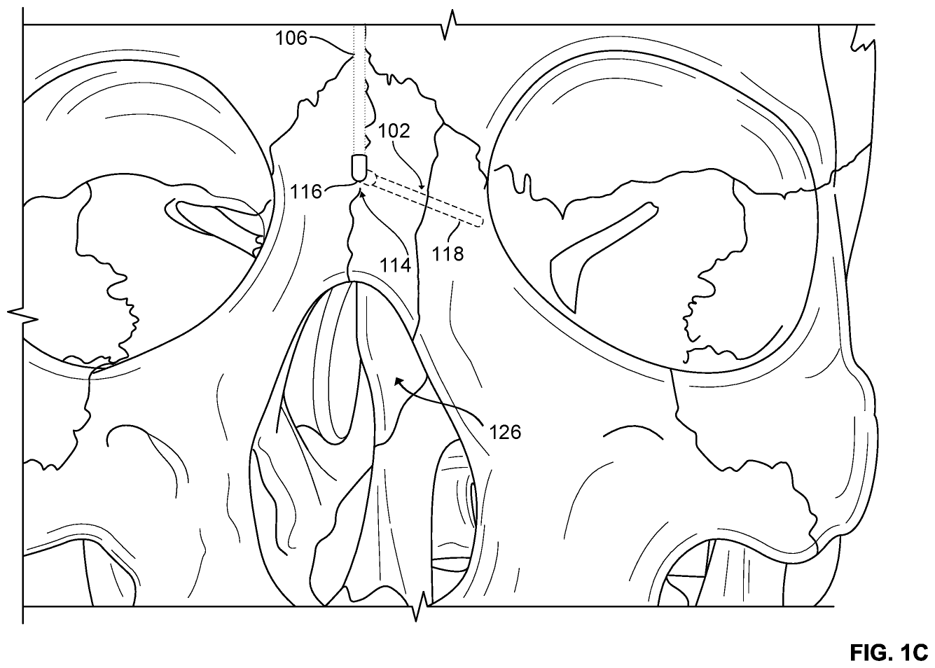

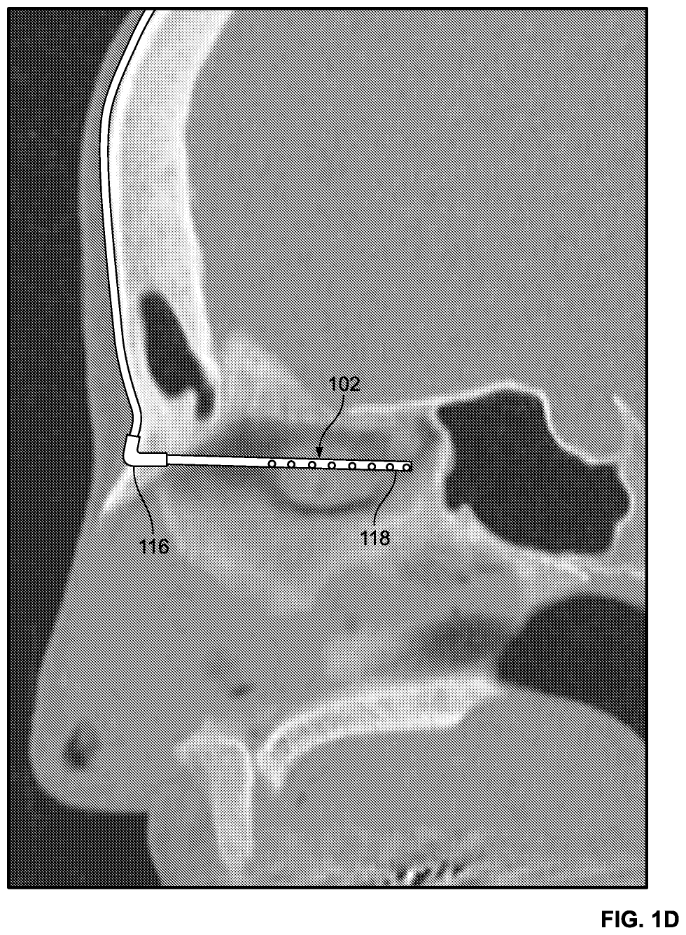

FIGS. 1A-1D illustrate an implantable catheter system for drug delivery 100 in the submucosal space of the olfactory mucosa 101 of the subject 110. As shown in FIG. 1A, the system 100 comprises an implantable device 102, a drug reservoir 104, and a tube 106 fluidly connecting the implantable device 102 to the drug reservoir 104. The drug reservoir 104 is implanted behind the ear 108 of the subject and the tube 106 is implanted extracranially along the skull 111 under the skin 112 of the subject 110. The implantable device 102 is positioned in the submucosal space of the olfactory mucosa 101 to deliver one or more therapeutic agents into the CNS via the olfactory bulb 122 (see FIG. 1B). The drug reservoir 104 is used to hold a drug or a therapeutic agent for delivery.

In FIG. 1B, the implantable device 102 is shown with a proximal portion 116 positioned in the nasion 114 (an anterior portion of the frontonasal suture that joins the nasal part of the frontal bone and the nasal bones) of the subject and a main body 120 of the implantable device positioned in the submucosal space of the olfactory mucosa. A portion of the implantable device (e.g., the proximal portion 116) can act as an anchor and/or a mechanical stop against tissue (e.g., nasion 114) during and/or after implantation of the device in the submucosal space to prevent undesired movement of the implantable device. Movement of the implantable device within the nasal region of the subject can also be limited by engaging a distal portion of the implantable device with tissue in the nasal region.

As depicted in FIG. 1B, the body 120 of the implantable device is an elongate tubular catheter. The body 120 includes a through hole opening at its distal end 118 and a plurality of apertures on its wall along its length to deliver drugs to the CNS. The apertures extend from the mid portion 124 and the distal portion 118 of the body 120 such that the apertures along the wall can deliver drugs to an area proximate to the olfactory nerves 128 and olfactory bulb 122. The body of the implantable device can be shaped in various forms and lengths, as desired. The apertures along the wall of the body can be sized and positioned in different locations, as desired.

In FIG. 1C, the distal portion 118 of the implantable device 102 is positioned to engage the ethmoid bone 126 and the proximal portion 116 of the implantable device 102 engages the nasion 114. The implantable device 102, however, can be configured to engage and/or extend through specific anatomy, as desired. For example, in some embodiments, a proximal portion 116 of the implantable device can protrude from and/or extend though the nasion 114. In some embodiments, distal portion 118 of the implantable device 102 can placed on a portion of the ethmoid bone 126, engage with a surgically altered section of the ethmoid bone 126 or its surrounding tissue, or be positioned adjacent to one side of the ethmoid bone 126.

As shown in FIG. 1D, a CT image of a subject's head is superimposed with a schematic illustration of an exemplary implantable device 102 placed in the nasal region. The implantable device 102 is shown with a proximal portion 116 positioned in the nasion of the subject and a distal portion 118 positioned in the submucosal space of the olfactory mucosa. A portion of the implant (e.g., a proximal portion 116) can be configured to bend at an angle (e.g., U-shape, or L-shape) to help conform the implant to particular anatomy. In some embodiments, a portion of the implant can be made with a flexible material that allows the implant to flex, bend, and tightly conform to the shape of the forehead bone structure. For example, a curved shape (e.g., U-shaped, L-shaped bend) can advantageously allow a portion of the implant to protrude from the nasion and sharply bend to conform with the forehead bone structures, which can help minimize or eliminate visible protrusions through the skin in the forehead area. In some embodiments, a portion of the implant can be preset to a curved shape so that once the device is implanted, the preset portion of the implant returns to its original curved form.

The system for drug delivery can include one or more drug refill ports for adding drug to the system. In some examples, the drug reservoir includes a drug refill port fluidly connected to the drug reservoir. In such instances, the drug refill port is configured to receive a drug from an external source and to provide a fluidic path to the drug reservoir. Alternatively, or in addition to, the pump includes a drug refill port through which the drug reservoir can be filled from an external source.

In some examples, systems for drug delivery include an implantable drug reservoir and/or an implantable pump. The drug can be pushed or pumped from a drug reservoir through the tubing (e.g., catheter) and the distal portion of the implantable device. The drug can be pumped from the reservoir to the implantable device. The drug can include a liquid or a low viscosity gel in accordance with certain implementations.

The implantable system described herein and portions thereof can be implanted in various area of the body, as desired. For example, in some embodiments, implantable drug reservoir and/or the implantable pump is implanted in the head (e.g., posterior or superior portions), the neck, chest, arm, back, or the abdominal wall of the subject. Alternatively, the drug reservoir and/or the pump is fluidly connected to the implant and externally attached to the body of the subject (e.g., worn as a vest).

### Implantable Devices

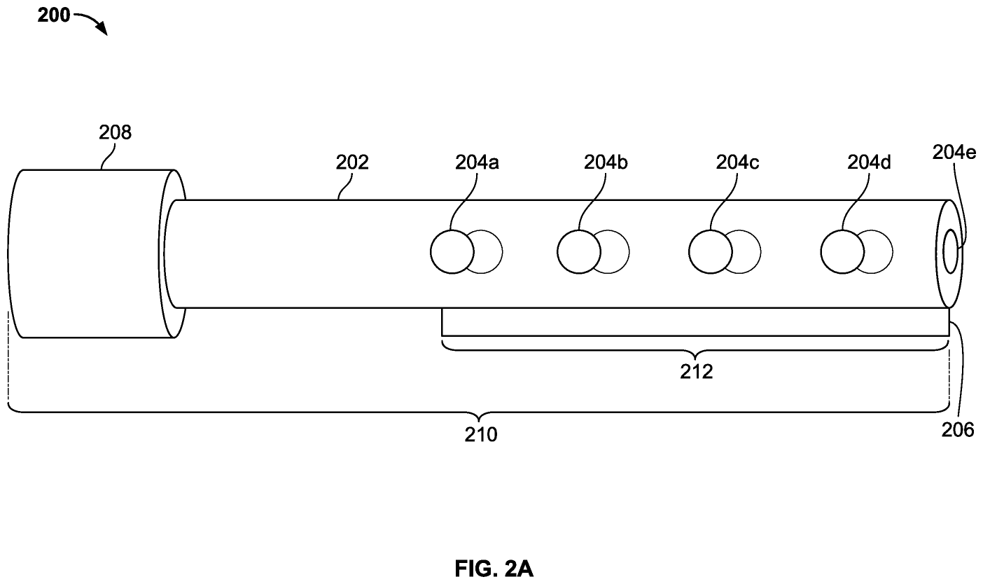

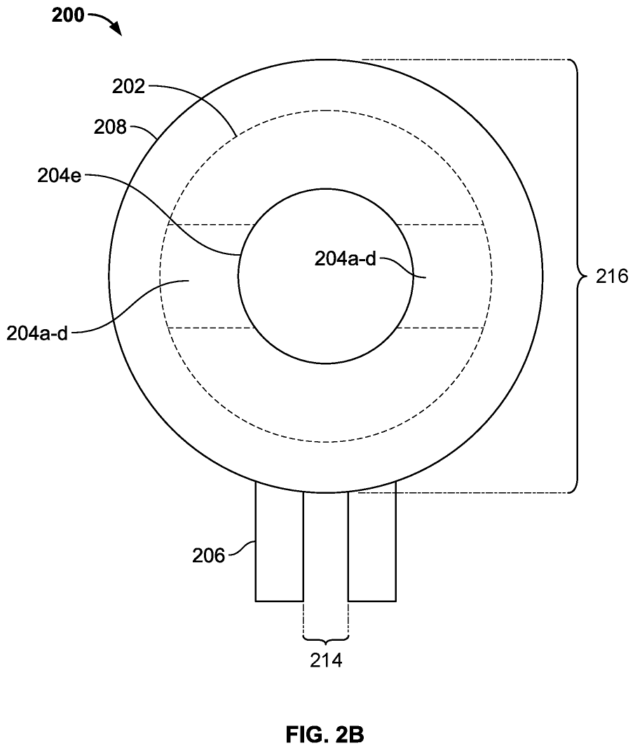

Aspects of the present disclosure provide implantable devices for continuous delivery of a drug into a nasal region of a subject. An example of an implantable device described herein is illustrated in FIGS. 2A-2B.

FIG. 2A shows a side view of an implantable device 200 as described herein. The implantable device 200 includes a distal body 202 having a length 210 and a pair of support members 206 having a length 212. The body 202 has a wall extending from a proximal end to a distal end, and a lumen extending therethrough 204e in a longitudinal direction. The body 202 comprises eight apertures 204a-204d that are located along the wall and that are in fluid communication with the lumen. The lumen and apertures provide a pathway for a drug to be moved through the implantable device to the nasal region of the subject. In some embodiments, the implantable device 200 includes a lumen that is sealed at the distal tip, and thus, does not include aperture 102e.

As shown in FIG. 2A, implantable device 200 includes a pair of support members 206 protruding from the body 202 in a transverse direction and extending along the body 202 in the longitudinal direction. In some examples, the implantable device comprises a reinforced section 208 (e.g., a collar) at the proximal end of the distal portion of the body 202. The reinforced section 208 can be a portion of the body having an increased thickness and/or flexibility (e.g., a decreased durometer) as compared to the remaining portions of the device body. As shown in FIG. 2A, the reinforced section is a straight tubular section. In some embodiments, the reinforced section 208 is preset with a predefined curved shape to help navigate and/or anchor the implant to its targeted implant location.

FIG. 2B shows a front view of the implantable device of FIG. 2A. The pair of support members 206 are separated by a gap distance 214 that extends along in the longitudinal direction. The gap distance 214 between the pair of support member can engage tissue during implantation to prevent movement and/or displacement of the implantable device within the nasal region. The implantable device has an outer diameter 216.

The support members can be sized and shape, as desired, to achieve proper orientation and securement to the implant location. For example, the pair of support members can include support members that are the same shape and size or a different shape and size. The support members can be integrally formed with the body or attached to the body. The support members can be positioned parallel or non-parallel to one another along the body of the implantable device. In some examples, the support members are substantially parallel to one another in the transverse direction. The support members can be positioned in a various directions along the body of the implantable device. In some examples, the support members extend in the same direction or in a different direction along a transverse plane.

The support members can be disposed in various positions along the body of the implantable device. For example, when the body has a circular cross-section along a transverse plane, the support members are disposed on a minor arc portion of the circular cross-section. In such instances, the minor arc portion of the circular cross-section is less than 25% of a circumference of the circular cross-section. Placement of the support members in close proximity (e.g., less than 25% of a circumference of the circular cross-section) may serve to advantageously form a groove-like spacing adjacent to the body of the implant. The groove-like spacing may be configured to receive tissue that helps to align and/or secure the implant at the target implant location.

The implantable device is dimensioned such that it fits comfortably within the nasal region (e.g., submucosal space) of a subject. Accordingly, the implantable device can be a variety of different shapes and sizes.

The distal body of the implantable device can have a variety of different shapes (e.g., conical frustrum-shaped, spherical, rectangular, tubular, or ellipsoidal).

The distal body can vary in size. In some examples, the distal body has a length 210 of about 30 mm to about 60 mm (e.g., about 35 mm to about 60 mm, about 40 mm to about 60 mm, about 45 mm to about 60 mm, about 50 mm to about 60 mm, about 55 mm to about 60 mm, about 30 mm to about 55 mm, about 30 mm to about 50 mm, about 30 mm to about 40 mm, or about 30 mm to about 35 mm).

In some examples, the distal body has an outer diameter 216 of about 0.1 mm to about 3 mm (e.g., about 0.25 mm to about 3 mm, about 0.5 mm to about 3 mm, about 0.75 mm to about 3 mm, about 1.0 mm to about 3 mm, about 1.5 mm to about 3 mm, about 2 mm to about 3 mm, about 2.5 mm to about 3 mm, about 0.1 mm to about 2.5 mm, about 0.1 mm to about 2 mm, about 0.1 mm to about 1.5 mm, about 0.1 mm to about 1.0 mm, about 0.1 mm to about 0.75 mm, about 0.1 mm to about 0.5 mm, or about 0.1 mm to about 0.25 mm).

The support members of the implantable device can have a variety of different shapes (e.g., elongate fin shape, rectangular shape along the transverse plane or along the longitudinal plane).

The support members can vary in size. In some examples, each of the support members has a length 212 of about 1 mm to about 10 mm, e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 8 mm to about 10 mm, about 9 mm to about 10 mm, about 1 mm to about 9 mm, about 1 mm to about 8 mm, about 1 mm to about 7 mm, about 1 mm to about 6 mm, about 1 mm to about 5 mm, about 1 mm to about 4 mm, about 1 mm to about 3 mm, or about 1 mm to about 2 mm.

The height to length ratio of the support members can vary. In some examples, each of the support members has a height to length ratio of about 0.1 to about 1.0, e.g., about 0.2 to about 1.0, about 0.3 to about 1.0, about 0.4 to about 1.0, about 0.5 to about 1.0, about 0.6 to about 1.0, about 0.7 to about 1.0, about 0.8 to about 1.0, about 0.9 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, or about 0.1 to about 0.2. The height to length ratio contributes to amount of surface area that directly engages with tissue. Thus, the height to length ratio can affect implant securement with the surrounding tissues and/or affect the stability of the implant over time.

The gap distance 214 between the pair of support members can vary in size. In some examples, the gap distance between the pair of support members is about 1 mm to about 3 mm, e.g., about 1.5 mm to about 3 mm, about 2 mm to about 3 mm, about 2.5 mm to about 3 mm, about 1 mm to about 2.5 mm, about 1 mm to about 2 mm, or about 1 mm to about 1.5 mm. The gap distance determines the volume of tissue that the implantable device will engage between the support members. Thus, the gap distance can impact implant securement and stability over time.

The implantable device comprises one or more apertures that are in fluid communication with the lumen, thereby providing an opening from which drug can exit the implantable device and enter the nasal region of the subject.

The one or more apertures can be located along the wall of the distal body, at the end of the distal body, or both along the wall and at the end of the distal body. The apertures can be located at various positions along the wall. In some examples, as shown in FIG. 2A, the apertures are positioned near the distal end of the body. In some examples, apertures are located along the wall at about 10 degrees to about 90 degrees, about 90 degrees, or about 90 degrees to about 180 degrees (e.g., from about 90 degrees to about 120 degrees, from about 120 degrees to about 160 degrees, or from about 140 degrees to about 170 degrees) from the support members. In some embodiments, the apertures along the wall can be directed to deliver one or more drugs in the direction of the olfactory nerves to increase the drug's therapeutic efficacy. The angle of the apertures relative to the support members can important in advantageously directing the delivery of the drugs to the desire location within the implanted region.

The size and number of aperture(s) in the implantable device can vary depending on the therapeutic agent being delivered. For example, the aperture has a diameter between about 20 μm and about 500 μm (e.g., about 100 μm and about 500 μm, about 200 μm and about 500 μm, about 300 μm and about 500 μm, about 400 μm and about 500 μm, about 20 μm and about 400 μm, about 20 μm and about 300 μm, about 20 μm and about 200 μm, about 20 μm and about 100 μm, or about 20 μm and about 50 μm). When an implantable device includes more than one aperture, the apertures can have the same size or a different size. The implantable device can include a single aperture or multiple apertures (e.g., 2, 3, 4, 5, 6, 7, 8, 9, 10, or more apertures). The aperture typically is circular in shape, although other shapes (e.g., oval, slot, polygonal, irregular shaped) are possible.

Any of the implantable devices described herein can comprise one or more additional components such as a guidewire lumen, an outer sheath, an endoscope, a dilation balloon, or a combination thereof.

### Methods for Implanting Implantable Devices

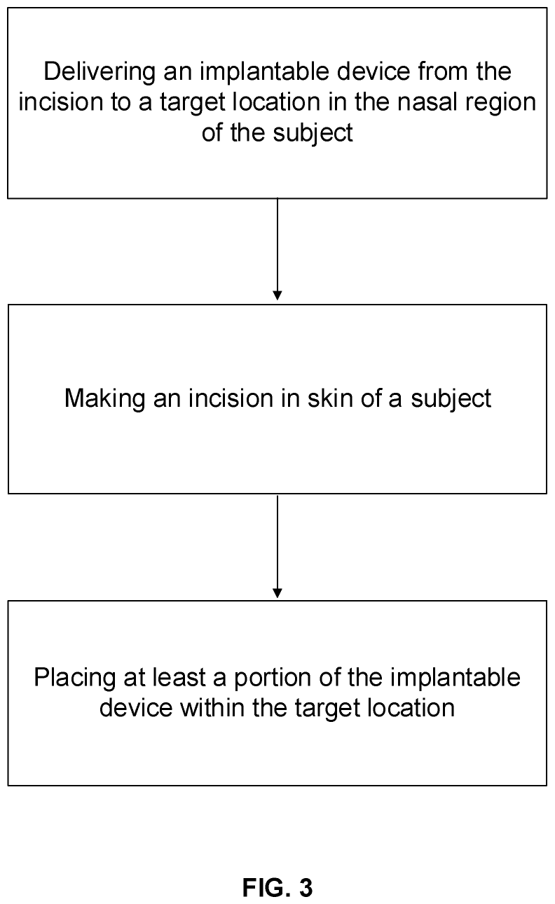

Also provided herein are methods for implanting any of the implantable devices described herein in a nasal region of a subject. As shown in FIG. 3, methods described herein comprise forming an incision in skin of a subject, passing the implantable device from the incision to a target location in a nasal region via a tunnel, and positioning at least a portion of the implantable device within the target location.

Methods described herein encompass forming at least one incisions in the subject (e.g., 1, 2, 3, 4, or more incisions). The incision in skin of the subject can be formed in any location suitable for passing the implantable device from the incision to another incision and/or to the target location in the nasal region of the subject. In some examples, an incision is formed in the occipital region of the subject (e.g., an incision in postauricular skin of the subject), the scalp of the subject (e.g., an incision in vertex scalp of the subject), and/or the intranasal regions of the subject.

Methods described herein include forming a tunnel extending from the incision to a target location in the nasal region of the subject. Any tunnel suitable for passing the implantable device from the incision to the target location can be used in methods described herein. In some examples, the tunnel is a subperiosteal tunnel. In some embodiments, the tunneling can be achieved using tunneling devices, such as tunneling wires and/or tunneling catheters. The tunneling devices may be used separately or together with the implantable system, or portions thereof during the implantation procedure.

The implantable device or portion thereof can be implanted in any target location in the nasal region of the subject suitable for delivery of the therapeutic agent to the subject's CNS. In some examples, a portion of the implantable device is implanted in the olfactory epithelium (e.g., the submucosal space of the olfactory epithelium).

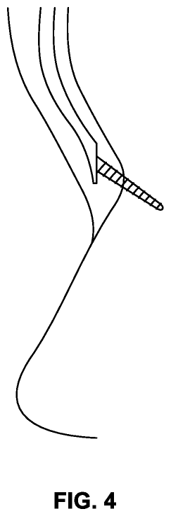

Methods described herein can include removing tissue from the subject. In some examples, methods described herein include removing connective tissue such as bone. For example, methods include drilling a hole that extends perpendicularly through a cranial bone (e.g., the nasion) of the subject, as shown in FIG. 4. As depicted in FIG. 4, the drilling can be accomplished by a subcutaneous drilling device that is introduced through the incision and advanced through the tunnel to a drilling location, which is near the target implant location. In some embodiments, the subcutaneous drilling device can be inserted through a guide catheter to the drilling location. In various embodiments, once a hole has been drilled through the cranial bone, a portion of the implantable device can be positioned in (e.g., the collar) and/or through the hole (e.g., distal tip with apertures).

In some examples, methods described herein include removing at least a portion of the ethmoid bone of the subject (e.g., a portion of the perpendicular plate of the ethmoid bone). In such instances, a portion of the implantable device (e.g., the support members) is positioned to engage the perpendicular plate of the ethmoid bone in the subject.

Methods described herein encompass implanting the implantable device and configuring the device for delivery of a therapeutic agent. For example, methods described herein include fluidly connecting the implant to a drug reservoir via a tube and implanting the drug reservoir and the tube in the subject. The drug reservoir can then be implanted in any suitable location (e.g., implanted in the head, the neck, or the abdominal wall of the subject).

### Treatment Methods

The implantable drug delivery devices and systems described herein can be used to continuously administer a therapeutic agent to a CNS of a subject in need thereof. Such methods comprise implanting the implantable device into the nasal region of the subject and continuously administering the therapeutic agent.

A subject to be treated by the methods described herein can be any subject for whom administration of a therapeutic agent via implantation of the implantable device is desired. For example, the subject who needs the treatment can be a subject having, suspected of having, or at risk for having a neurological disorder. Any neurological disorder can be treated using any of the implantable devices described herein. Non-limiting examples of neurological disorders include Parkinson's disease, Alzheimer's disease, a brain cancer (e.g., glioblastoma multiforme, oligodendroglioma, astrocytoma, oligoastrocytoma, ependymoma, medulloblastoma, or meningioma), Huntington's disease, Bell's palsy, stroke, epilepsy, migraine, a sleep disorder, multiple sclerosis, muscular dystrophy, amyotrophic lateral sclerosis, encephalitis, Creutzfeldt-Jakob disease, meningitis, frontotemporal dementia, schizophrenia, and depression.

A subject having, suspected of having, or at risk for having a neurological disorder can be identified by routine medical examination, e.g., laboratory tests (e.g., blood tests), magnetic resonance imaging (MRI), computerized tomography (CT) scans, electromyogram (EMG), nerve conduction studies, biopsy (e.g., muscle biopsy), spinal tap, and cerebral angiogram.

A subject having, suspected of having, or at risk for having a neurological disorder might show one or more symptoms of a neurological disorder, e.g., headache, difficulty walking and/or speaking, slurred speech, difficulty swallowing, muscle cramps and/or twitching, muscle atrophy, tremor, vision problems, paralysis or numbness, weakness, cognitive or behavioral changes, impaired posture and balance, loss of consciousness, nausea, convulsions or seizures, and dilation of one or both pupils of the eyes.

A subject at risk for having a neurological disorder can be a subject having one or more of the risk factors for that neurological disorder. For example, the risk factors associated with certain neurological disorders include a family history of a neurological disorder, age, sex, genetic abnormalities, exposure to a toxin, exposure to a virus, and a combination thereof.

An effective amount of a therapeutic agent can be administered to a subject (e.g., a human) in need of the treatment via implantation of the implantable device into the nasal region of the subject and administration of the therapeutic agent to the CNS of the subject through the implantable device.

Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. Empirical considerations such as the half-life of an agent will generally contribute to the determination of the dosage.

Alleviating a neurological disorder includes delaying the development or progression of the disease, and/or reducing disease severity. Alleviating the disease does not necessarily require curative results.

Treatment methods described herein encompass delivering any therapeutic agent including small molecules, peptides, proteins (e.g., antibodies), and nucleic acids (e.g., mRNA, siRNA, antisense oligonucleotides).

The therapeutic agent can be any size. In some embodiments, the therapeutic agent has a molecular size of greater than 500 Da (e.g., greater than 600 Da, 700 Da, 800 Da, 900 Da, 1 kDa, 2 kDa, 3 kDa, 4 kDa, 5 kDa, 10 kDa, 20 kDa, 30 kDa, 70 kD, or 100 kD). The therapeutic agent can be charged or uncharged. In some embodiments, the therapeutic agent has a net positive or net negative charge. In some embodiments, the therapeutic agent that is a polar molecule.

In some embodiments, the therapeutic agent is a chemotherapeutic agent. Non-limiting examples of chemotherapeutic agents include cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab.

In some embodiments, the therapeutic agent is an anti-depressant agent. Non-limiting examples of anti-depressant agents include selective serotonin reuptake inhibitors (e.g., citalopram, escitalopram, fluoxetine, paroxetine, or sertraline), serotonin-norepinephrine reuptake inhibitors (e.g., desvenlafaxine, duloxetine, milnacipran, and venlafaxine), noradrenergic and specific serotonergic antidepressants (e.g., mianserin and mirtazapine), norephinephrine reuptake inhibitors (e.g., atomoxetine, mazindol, reboxetine, and viloxazine), norepinephrine-dopamine reuptake inhibitors (e.g., bupropion), selective serotonin reuptake enhancers (e.g., tianeptine), norephinephrine-dopamine disinhibitors (e.g., agomelatine), tricyclic antidepressants (e.g., amitriptyline, clomipramine, doxepin, imipramine, and trimipramine), secondary amine tricyclic depressants (e.g., desipramine, nortriptyline, and protripyline), monoamine oxidase inhibitors (e.g., isocarboxazid, moclobemide, phenelzine, selegiline, and tranylcypromine), buspirone, gepirone, nefazodone, trandospirone, trazodone, bupropion, benzodiazepines, amphetamine, methylphenidate, modafinil, lithium, carbamazepine, sodium valproate, and lamotrigine.

In some embodiments, the therapeutic agent is an anti-psychotic agent. Non-limiting examples of anti-psychotic agents include risperidone, olanzapine, and quetiapine.

In some embodiments, more than one therapeutic agent or a combination of therapeutic agents can be administered to a subject in need of the treatment. Alternatively, or in addition to, the therapeutic agent can be used in conjunction with other agents that serve to enhance and/or complement the effectiveness of the therapeutic agent.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

In order that the invention described may be more fully understood, the following example is provided. The example described in this application is offered to illustrate the devices, systems, and methods provided herein and is not to be construed in any way as limiting.

### Example 1: In Vivo Peptide Delivery in Rat Brain Sub-Regions

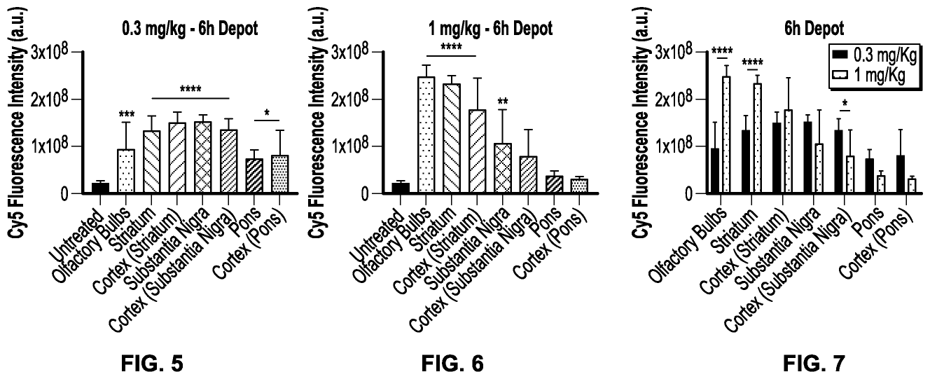

This Example examined CNS delivery of a fluorescently labeled BBB-impermeant peptide via the minimally invasive nasal depot (MIND) technique. Healthy Sprague Dawley rats were administered Cy5.5-labeled peptide (0.3 mg/kg or 1 mg/kg) in a loaded osmotic core-shell depot implanted using the MIND technique. After 6 hours, rats were sacrificed and rat brains and olfactory bulbs were harvested. Tissue sections were fixed and delivery was calculated by measuring intensity of fluorescent staining over the tissue section. Untreated rats and tissue sections harvested therefrom were used as a negative control. Statistically significant uptake of the fluorescently labeled peptide was observed at both the 0.3 mg/kg dose (FIG. 5) and the 1.0 mg/kg dose (FIG. 6). A two-way ANOVA test was performed to examine the uptake of the two doses at each region of the brain. As shown in FIG. 7, uptake in certain tested regions was higher for the higher dose (e.g., olfactory bulbs) while other regions showed higher uptake at the lower dose (e.g., pons, corex (pons)).

Taken together, the results provided herein demonstrate that a therapeutic agent such as peptide can be effectively delivered to the CNS by intranasal administration.

### Example 2: In Vivo Distribution of BDNF AT Levels in Rat Brain Sub-Regions

Brain derived neurotrophic factor (BDNF) is a neurotrophin important for the survival, differentiation and maturation of neurons of the nervous system (Huang & Reichardt, 2001; Padmakumar, et al., 2020). Reduced expression of BDNF in the nigrostriatal dopaminergic brain regions has been implicated in the pathogenesis of Parkinson's disease (Parain, et al., 1999; Palasz, et al., 2020; Mogi, et al., 1999; Murer, et al., 2001).

BDNF AntagoNATs are single stranded short synthetic oligonucleotide-based compounds possessing the ability to inhibit BDNF-AS, the conserved noncoding natural antisense transcript (NAT) which normally slows down BDNF sense RNA transcription and represses endogenous BDNF protein production. It has been previously reported that BDNF AntagoNAT (BDNF AT) treatment can induce BDNF mRNA and protein upregulation and in vitro differentiation and in vivo proliferation of neuronal cells (Modarresi, et al., 2012). Nevertheless, the BBB-impermeant nature of such ATs typically requires invasive modes of CNS delivery such as intrathecal (IT) or intracerebroventricular (ICV) routes wherein the BBB is physically breached (Khorkova & Wahlestedt, 2017).

This Example compared CNS delivery of BDNF AT using the following approaches: ICV, intravenous (IV), and intranasal administration via the minimally invasive nasal depot (MIND) technique. Healthy Sprague Dawley rats (n=4) were administered BDNF AT (0.15 mg/kg) by ICV, IV, or a BDNF AT loaded osmotic core-shell depot implanted using the MIND technique. After delivery of BDNF AT for 2 hours, rats were sacrificed and rat brains and olfactory bulbs were harvested. Tissue samples from striatum, hippocampus, substantia niagra and cerebellum were retrieved using tissue biopsy punches. The BDNF AT levels in these tissue extracts were quantified by AT hybridization assay as described previously (Padmakumar, et al., 2021).

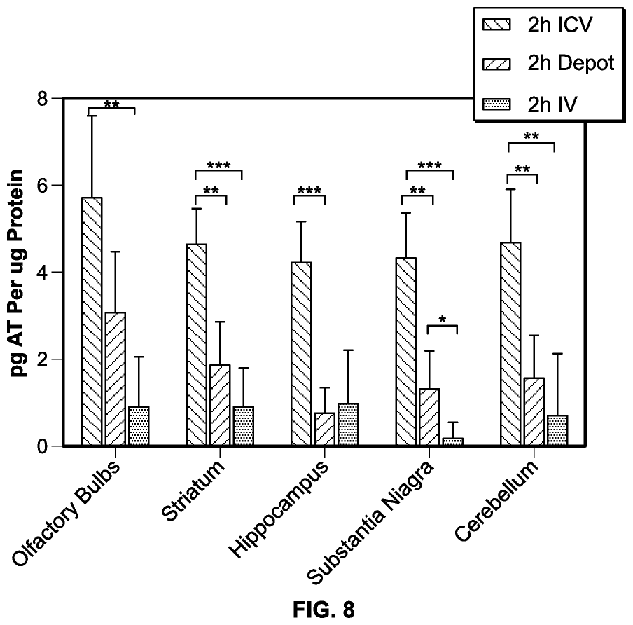

As shown in FIG. 8, rats administered BDNF AT using the MIND technique displayed increased uptake in most of the brain regions examined compared to rats administered BDNF AT using IV. Importantly, increased uptake was pronounced in the substantia niagra region, which is a key target in Parkinson's disease due to reduced expression of BDNF in the nigrostriatal dopaminergic brain regions in patients with Parkinson's disease. BDNF uptake levels were lower for rats administered BDNF AT using the MIND technique compared to uptake levels in rats administered BDNF AT via ICV (FIG. 8).

Taken together, the results provided herein demonstrate that a BBB-impermeant therapeutic agent such as BDNF AT can be effectively delivered to the CNS by intranasal administration.

### Example 3: Exemplary Surgical Procedure in a Human

This Example describes the implantation of an implantable device in a subject.

After standard prepping and draping, a semi-circular incision was placed in the post-auricular skin in the occipital region of the subject. The post-auricular scalp was then incised and dissected down to the cranial bone. A second small access incision was made in the midline of the vertex of the head around the junction of the frontal and parietal bone suture lines. A subperiosteal tunnel was formed between the post-auricular incision and the midline incision using a curved tunneling device.

A curved tunneling device as then used to create a subperiosteal tunnel from the midline incision to the nasion. Tunneling can be performed using palpation and/or endoscopic guidance. The curved tunneling device allows adequate subperiosteal dissection around the curved frontal bone. A device with a right angle drill is used to drill a hole that extends perpendicularly through a nasion of the subject. The inner diameter of the hole in the nasion was drilled to match the outer diameter of the reinforced section (e.g., collar) of the implantable device to allow the reinforced section to sit in the hole. The reinforced section prevents kinking of the body of the implantable device and maintains a nasionotomy.

Endoscopically, a submucoperichondrial flap was elevated to expose the olfactory nerves within the olfactory neuroepithelial space. A segment of the superior aspect of the perpendicular plate of the ethmoid bone was removed to enable bilateral drug access and to provide a shelf upon which the implantable device can sit to avoid migration. The mucosal incision was carried superiorly over the nasal vault to expose the bone of the nasal beak. A high speed drill was used to thin out the bone of the beak and to expose the periosteum of the nasion. The procedure described above can be performed with the assistance of a frameless stereotactic image guidance system.

The implantable device was then tunneled from the post-auricular incision to the midline incision. After securing the implantable device, a surgical device was used to pass the implantable device under the frontal subperiosteal tunnel and through the reinforced section (e.g., collar).

The implantable device was then endoscopically retrieved and placed into the submucosal space of the olfactory neuroepithelium under direct visualization to ensure that the implantable device was passed to the most posterior visualized olfactory nerve and that there was no tension or minimal tension between the implantable device and the reinforced section. In order to prevent inferior migration of the implantable device, the implantable device was placed so that the support members straddle the free edge of the perpendicular plate of the ethmoid bone. The mucosal flap were then closed by suture.

The proximal end of the body of the implantable device was then connected to a tube, which is connected to a drug reservoir. The drug reservoir was secured directly to the bone using self-tapping screws or a suture applied through holes drilled through the outer calvarial table.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An implantable device for drug delivery, the implantable device comprising:
a distal body having a wall extending from a proximal end to a distal end, and a lumen extending therethrough in a longitudinal direction, the distal body comprising one or more apertures located along the wall, the apertures being in fluid communication with the lumen; and
a pair of support members protruding from the body in a transverse direction and extending along the body in the longitudinal direction, wherein the support members are separated by a gap distance that extends along in the longitudinal direction.

2. The implantable device of claim 1, wherein the support members are substantially parallel to one another in the transverse direction.

3. The implantable device of claim 1, wherein the support members extend in the same direction along a transverse plane.

4. The implantable device of claim 1, wherein the distal body has a circular cross-section along a transverse plane, and wherein the support members are disposed on a minor arc portion of the circular cross-section.

5. The implantable device of claim 4, wherein the minor arc portion of the circular cross-section is less than 25% of a circumference of the circular cross-section.

6. The implantable device of claim 1, wherein the support members have a rectangular shape along the transverse plane.

7. The implantable device of claim 1, wherein the support members have a rectangular shape along the longitudinal plane.

8. The implantable device of claim 1, wherein the support members is an elongate fin.

9. The implantable device of claim 1, wherein the gap distance is configured to engage tissue during implantation.

10. The implantable device of claim 1, wherein each support member has a height to length ratio of about 0.1 to 1.0.

11. The implantable device of claim 1, wherein the apertures are located along the wall at about 90 degrees from the support members.

12. The implantable device of claim 1, wherein the apertures are located along the wall from about 90 degrees to about 180 degrees from the support members.

13. The implantable device of claim 1, wherein the apertures are located along the wall from about 10 degrees to no more than 90 degrees from the support members.

14. The implantable device of claim 1, wherein the body has a length of about 30 mm to about 60 mm.

15. The implantable device of claim 1, wherein the body has an outer diameter of about 1 mm to about 3 mm.

16. The implantable device of claim 1, wherein each of the support members has a length of about 1 mm to about 10 mm.

17. The implantable device of claim 1, wherein the gap distance between the pair of support members is about 1 mm to about 3 mm.

18. The implantable device of claim 1, comprising a collar coupled at the proximal end of the distal portion, wherein the collar has a wall thickness that is greater than a wall thickness of the distal portion.

19. A system for drug delivery, the system comprising:
the implantable device of claim 1;
a drug reservoir; and
a tube fluidly connecting the implant to the drug reservoir.

20. The system of claim 19, wherein the drug reservoir comprises a drug refill port fluidly connected to the drug reservoir, the drug refill port configured to receive a drug from an external source and to provide a fluidic path to the drug reservoir.

21. The system of claim 19, wherein the drug reservoir is an implantable drug reservoir.

22. The system of claim 19, comprising a pump communicably coupled to the drug reservoir and configured to move a drug from the drug reservoir to the implant via the tube.

23. The system of claim 22, wherein the pump comprises an additional drug refill port through which the drug reservoir can be filled.

24. The system of claim 22, wherein the pump is an implantable pump.

25. A method for implanting an implantable device in a nasal region, the method comprising:
making an incision in skin of a subject;
delivering the implantable device of claim 1 from the incision to a target location in the nasal region; and
placing at least a portion of the implantable device within the target location.

26. The method of claim 25, comprising forming a tunnel extending from the incision to a target location in the nasal region of the subject, wherein the forming the tunnel includes drilling a hole that extends perpendicularly through a nasion of the subject.

27. The method of claim 25, comprising fluidly connecting the implant to a drug reservoir via a tube and implanting the drug reservoir and the tube in the subject, wherein the drug reservoir is implanted in the head, the neck, or the abdominal wall of the subject.

28. The method of claim 27, continuously delivering one or more drugs from the drug reservoir to the implantable device.

29. The method of claim 25, the target location includes a submucosal space of an olfactory mucosa of the subject.
